(19) United States
(12) Patent Application Publication
(10) Pub. No.: US 2022/0133728 A1
(43) Pub. Date: May 5, 2022

(54) METHODS FOR TREATING VITILIGO LESIONS HAVING IMPROVED EFFICACY

(71) Applicant: ARCUTIS BIOTHERAPEUTICS, INC., Westlake Village, CA (US)

(72) Inventors: Robert HIGHAM, Westlake Village, CA (US); David BERK, Westlake Village, CA (US); Frank WATANABE, Westlake Village, CA (US)

(73) Assignee: ARCUTIS BIOTHERAPEUTICS, INC., Westlake Village, CA (US)

(21) Appl. No.: 17/513,199

(22) Filed: Oct. 28, 2021

Related U.S. Application Data
(60) Provisional application No. 63/106,983, filed on Oct. 29, 2020.

(51) Int. Cl. A61K 31/519; A61P 17/00; A61K 9/00; A61K 47/20; A61K 47/10; A61K 47/34; A61K 47/32; A61K 47/12; A61K 47/14; A61K 47/36; A61K 47/02; A61N 5/06

(52) U.S. Cl. CPC A61K 31/519; A61P 17/00; A61K 9/0014; A61K 47/20; A61K 47/10; A61K 47/34; A61N 2005/0661; A61K 47/12; A61K 47/14; A61K 47/36; A61K 47/02; A61N 5/0616; A61N 5/062; A61K 47/32

(57) ABSTRACT

Methods for treating vitiligo lesions including administering to a patient having vitiligo lesions a combination of a topical treatment and ultraviolet B (“UVB”) phototherapy. As disclosed herein, the inventors of the present invention have made the surprising discovery that the JAK inhibitor SHR0302 applied topically combined with narrowband UVB phototherapy dramatically repigments vitiligo lesions, including vitiligo lesions of the face and neck.

### CROSS REFERENCE TO RELATED APPLICATIONS

The present application claims priority to U.S. Provisional Application No. 63/106,983 filed on Oct. 29, 2020, the disclosure of which is incorporated herein in its entirety by reference.

### FIELD OF THE INVENTION

The subject matter disclosed herein generally relates to methods for treating vitiligo lesions including administering to a patient having vitiligo lesions a combination of a topical treatment and ultraviolet B (“UVB”) phototherapy. As disclosed herein, the inventors of the present invention have made the surprising discovery that the JAK inhibitor SHR0302 applied topically combined with narrowband UVB phototherapy dramatically repigments vitiligo lesions, including vitiligo lesions of the face and neck.

### BACKGROUND OF THE INVENTION

Vitiligo is a common autoimmune disease affecting 0.5% to 2% of the world's population. Although generally more prominent in patients with darker skin types, vitiligo affects all races and both sexes. Vitiligo often presents as acquired, small, asymmetrical lesions that tend to increase in size over time and become more numerous, coalescing into larger areas. Although vitiligo can occur on any area of the body, the face is commonly affected.

Vitiligo is associated with a significant burden of disease, greatly affecting a patient's quality of life similar to that seen in psoriasis and atopic dermatitis (Linthorst 2009). Vitiligo can be particularly bothersome and disfiguring when it affects the face and neck as these areas are visible and may be difficult to camouflage (Elbuluk 2017). Numerous studies have demonstrated that quality of life and mental health are more negatively impacted in patients with vitiligo on the face and other visible areas (Elbuluk 2017, Amer 2016, Eleftheraidou 2012). The negative impact of face/head/neck on quality of life has been reported to be independent of the extent of disease (Bhandarkar 2012). The impact of vitiligo may extend to negative treatment by others, with impact on professional prospects and relationships. With respect to treatment, survey data from patients and providers has identified repigmentation specifically of the face and hands as particularly important (Eleftheriadou 2012).

There are currently no FDA-approved topical treatments for repigmentation of vitiligo. There is an unmet need for novel compounds, therapeutic strategies, and combination therapies for vitiligo in order to better treat this common disease, which significantly affects a patient's quality of life. Better treatments are particularly needed for facial vitiligo given its profound impact on patients.

### SUMMARY OF THE INVENTION

The present invention relates to methods for treating vitiligo lesions including administering to a patient having vitiligo lesions a combination of a topical treatment and ultraviolet B (“UVB”) phototherapy. As disclosed herein, the inventors of the present invention have made the surprising discovery that the JAK inhibitor SHR0302 applied topically combined with narrowband UVB phototherapy dramatically repigments vitiligo lesions, including vitiligo lesions of the face and neck. In accordance with one embodiment of the invention, it has surprisingly been discovered that the JAK inhibitor SHR0302 applied topically combined with narrowband UVB phototherapy dramatically repigments vitiligo lesions, including but not limited to vitiligo lesions of the face, neck, forearms, elbows, and hands. In preferred embodiments, the JAK inhibitor SHR0302 applied topically combined with narrowband UVB phototherapy repigments vitiligo lesions of the face and neck.

In certain embodiments of the present invention, a method of treating vitiligo in a subject in need thereof is provided. The method includes topically administering to the subject a therapeutically effective amount of SHR0302 or a salt thereof. The method further includes administering to the subject an ultraviolet B phototherapy.

In certain embodiments, the therapeutically effective amount of SHR0302 is administered in a topical pharmaceutical composition. The topical pharmaceutical composition can include SHR0302 as a free base or pharmaceutically acceptable salt. The pharmaceutical composition can comprise about 0.10% w/w to about 0.50% w/w of SHR0302. The pharmaceutical composition can further comprise a solvent. The pharmaceutical composition can further comprise an antioxidant, a preservative, an emulsifier, a moisturizer, or a thickener. In certain embodiments, the pharmaceutical composition is selected from the group consisting of an oil-in-water emulsion, a water-in-oil emulsion, a microemulsion, a nanoemulsion, a foam, a spray, a hydrophilic ointment, or a hydrophobic ointment.

In certain embodiments, the pharmaceutical composition is topically administered at least once daily for at least one week. In other embodiments, the pharmaceutical composition is administered twice daily for at least one week.

In certain embodiments, the subject having vitiligo is administered UVB phototherapy. In preferred embodiments, the UVB phototherapy is narrowband UVB phototherapy. The ultraviolet B phototherapy can be administered to the subject between one and three times per week.

The methods of the present invention can further include treatment of vitiligo lesions, including but not limited to treating patients having vitiligo lesions of the face, neck, forearms, and/or hands. The method includes topically administering to the subject a therapeutically effective amount of SHR0302 or a salt thereof. The method further includes administering to the subject an ultraviolet B phototherapy. The methods described herein can alleviate the symptoms of vitiligo, including repigmenting a subject's vitiligo lesions. The methods described herein can further reduce the subject's vitiligo area and severity index (VASI). VASI scores may designate a specific anatomical region, for example facial vitiligo area and severity index (F-VASI), face and neck vitiligo area and severity index (F/N-VASI), forearm vitiligo area and severity index (FOREARM-VASI).

The methods of the present invention can further include repigmenting vitiligo lesions, including vitiligo lesions in the face and neck, of a subject displaying said lesions. The method includes topically administering to the subject a therapeutically effective amount of SHR0302 or a salt thereof. The method further includes administering to the subject an ultraviolet B phototherapy.

### DETAILED DESCRIPTION OF THE INVENTION

Before the present invention is described in detail below, it is to be understood that this invention is not limited to the particular methodology, protocols, and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art to which this invention belongs.

All publications, patents and patent applications cited herein are hereby incorporated by reference in their entirety unless otherwise stated. Where the same term is defined in a publication, patent, or patent application and the present disclosure incorporated herein by reference, the definition in the present disclosure represents a controlling definition. For publications, patents and patent applications referenced to describe a particular type of compound, chemistry, etc., the portion relating to such compounds, chemistry, etc. is the portion of the literature incorporated herein by reference.

Note that as used herein, the singular forms “a,” “an,” and “the” include plural referents unless the context clearly dictates otherwise. Thus, for example, “active ingredient” includes a single ingredient and two or more different ingredients, “solvent” refers to a single solvent and two or more different solvents or a complex mixture of solvents, and “sulfate salt” includes a single sulfate salt as well as two or more different sulfate salts.

The term “about” when used in connection with a numerical value is meant to encompass numerical values within a range having a lower limit that is 5% smaller than the indicated numerical value and having an upper limit that is 5% larger than the indicated numerical value.

“Pharmaceutically acceptable” means generally safe for administration to humans or animals. Preferably, a pharmaceutically acceptable component is one that has been approved by a regulatory agency of the Federal or a state government or listed in the U.S. Pharmacopeia, published by the United States Pharmacopeial Convention, Inc., Rockville Md., or other generally recognized pharmacopeia for use in animals, and more particularly in humans.

A “pharmaceutical composition” according to the invention may be present in the form of a composition, wherein the different active ingredients and diluents and/or carriers are admixed with each other, or may take the form of a combined preparation, where the active ingredients are present in partially or totally distinct form. An example for such a combination or combined preparation is a kit-of-parts.

The term “phototherapy” as used herein refers to controlled and/or prescribed application of light to an area of or the entire body of a patient in order to derive a therapeutic benefit. The term “phototherapy” includes deliberate or intentional exposure of the body to sunlight, however, exposure to light occurring during the course of ordinary light is not within this term.

As used herein, the terms “subject” “or patient” most preferably refers to a human being. The terms “subject” or “patient” may include any mammal that may benefit from the compounds described herein.

A “therapeutic amount” or “therapeutically effective amount” is an amount of a therapeutic agent sufficient to achieve the intended purpose. The effective amount of a given therapeutic agent will vary with factors such as the nature of the agent, the route of administration, the size of the subject to receive the therapeutic agent, and the purpose of the administration. The effective amount in each individual case may be determined empirically by a skilled artisan according to established methods in the art.

The term “topical” with respect to administration of a drug or composition refers to the application of such drug or composition to the epithelial surface outside the body, including the skin or cornea. For this application, application to the inside of a body opening such as the mouth, nose or ear is not considered a topical application.

As used herein, “treat,” “treating,” or “treatment” of a disease or disorder means accomplishing one or more of the following: (a) reducing the severity and/or duration of the disorder; (b) limiting or preventing development of symptoms characteristic of the disorder(s) being treated; (c) inhibiting worsening of symptoms characteristic of the disorder(s) being treated; (d) limiting or preventing recurrence of the disorder(s) in patients that have previously had the disorder(s); and (e) limiting or preventing recurrence of symptoms in patients that were previously symptomatic for the disorder(s).

As used herein, the terms “ultraviolet light” or “UV” refers to light with a wavelength between 10 and 400 nm, including but not limited to, ultraviolet B (UVB, 280-320 nm) and ultraviolet A (UVA, 320-400 nm) and narrow regions thereof, e.g., narrowband ultraviolet B (nbUVB, 311-312 nm) and UVA1 (340-400 nm), with any intensity or from any source, including a manufactured device or the sun.

The abbreviation “w/w” represents the relative concentration of the components in the composition as “weight to weight” (i.e., percentage refers to percentage of total weight), rather than based on volume or other quantities.

The present invention relates to methods for treating vitiligo lesions including administering to a patient having vitiligo lesions a combination of a topical treatment and ultraviolet B (“UVB”) phototherapy. As disclosed herein, the inventors of the present invention have made the surprising discovery that the JAK inhibitor SHR0302 applied topically combined with narrowband UVB phototherapy dramatically repigments vitiligo lesions. In accordance with one embodiment of the invention, it has surprisingly been discovered that the JAK inhibitor SHR0302 applied topically combined with narrowband UVB phototherapy dramatically repigments vitiligo lesions of the face and neck.

Vitiligo is characterized by a T helper type 1 (Th1)-mediated immune response with increased expression of interferon (IFN)-γ and chemokines C-X-C Motif Chemokine Ligand (CXCL) 9 and CXCL10 which impair the normal function of melanocytes and produce areas of depigmentation on the skin. Importantly, after IFN-γ binds to its cell surface receptor (IFNGR), signaling is through Janus Kinases (JAKs), especially JAK1 and JAK2. JAK1 is also an important mediator of signaling in the IL-15 pathway, which is strongly implicated in vitiligo. As such, JAK inhibition represents an effective strategy for the treatment of vitiligo by decreasing INF-γ signaling and downstream chemokine expression. Topical JAK inhibitors are particularly well suited for treating vitiligo given the anticipated lower systemic exposures, improved safety profile, and more direct cutaneous delivery offered with topical rather than oral administration of JAK inhibitors. Topical JAK inhibition is also efficacious in the treatment of vitiligo. In particular, topical JAK inhibition has demonstrated efficacy for vitiligo on the face, even more so compared to vitiligo elsewhere on the body.

The methods of the present invention include administering a topical JAK inhibitor to a subject having vitiligo, including a subject exhibiting vitiligo lesions, including vitiligo lesions of the face and neck. In the present invention, the JAK1 inhibitors are those disclosed in U.S. Pat. No. 9,527,851, which is hereby incorporated by reference. In a particularly preferred embodiment, the JAK1 inhibitor is (3aR,5S,6aS)—N-(3-methoxyl-1,2,4-thiadiazol-5-yl)-5-(methyl(7H-pyrrolo[2,3-d]pyrimidin-4-yl)amino)hexahydrocyclopenta[c]pyrrole-2(1H)-carboxamide, which is also known as SHR0302 or ARQ-250. The terms SHR0302 and ARQ-250 are used interchangeably herein. The structure of SHR0302 is:

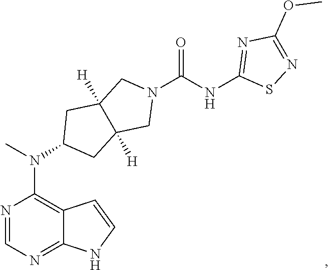 [SMILES: COc1nsc(NC(=O)N2C[C@H]3C[C@H](N(C)c4ncnc5[nH]ccc45)C[C@H]3C2)n1]

SHR0302 is a potent small molecule inhibitor of JAK 1 that has been shown to have a high selectively for JAK1 over JAK2, and thus has the potential to treat inflammatory diseases without causing the hematopoietic adverse effects, such as anemia, thrombocytopenia, and neutropenia, associated with JAK2 inhibition.

In the methods of the present invention, SHR0302 can be administered in a topical pharmaceutical composition. The pharmaceutical composition can include SHR0302 as a free base or a pharmaceutically acceptable salt. In certain embodiments of the claimed invention, the pharmaceutical composition includes the sulfate salt of SHR0302. Other suitable pharmaceutically acceptable salts can be found in Remington's Pharmaceutical Sciences, 17^{th} ed., Mack Publishing Company (1985), which is incorporated by reference herein.

Without meaning to be bound by theory, the efficacy of topical JAK inhibition is believed to be due to a high density of follicular units on the face relative to other parts of the body. Pigmented hair follicles are thought to be important reservoirs of melanocytes for repigmentation due to immune privilege of the follicle and melanocyte stem cells residing in the bulge region of follicles. As disclosed herein, the inventors have made the surprising discovery that the JAK inhibitor SHR0302 applied topically combined with narrowband UVB phototherapy dramatically repigments vitiligo lesions, including vitiligo lesions of the face and neck. The rationale for this combined approach is that a two-step strategy is needed, including JAK inhibition to suppress the local inflammation that contributes to disease, and ultraviolet light therapy to stimulate melanocytes to repigment lesions. Indeed, the present combination of topical SHR0302 combined with narrowband ultraviolet B (NB-UVB) is especially beneficial for facial vitiligo. The combination of JAK inhibition, particularly with topical SHR0302, combined with narrowband UVB phototherapy is believed to facilitate more complete and rapid repigmentation relative to JAK inhibition alone and phototherapy alone.

In the present invention, SHR0302 is administered topically. The pharmaceutical composition can include SHR0302 as a free base or a pharmaceutically acceptable salt. The topical pharmaceutical composition can be in many different forms including, for example, solutions, liquids, sprays, foams, lotions, gels, and the like. Preferably, SHR0302 is administered in a topical pharmaceutical composition in one of the following forms:

An oil-in-water emulsion: The product may be an emulsion comprising a discrete phase of a hydrophobic component and a continuous aqueous phase that includes water and optionally one or more polar hydrophilic excipients as well as solvents, co-solvents, salts, surfactants, emulsifiers, and other components. These emulsions may include water-soluble or water-swellable polymers that help to stabilize the emulsion.

A water-in-oil emulsion: The compositions may be an emulsion that includes a continuous phase of a hydrophobic component and an aqueous phase that includes water and optionally one or more polar hydrophilic carrier(s) as well as salts or other components. These emulsions may include oil-soluble or oil-swellable polymers as well as one or more emulsifier(s) to help to stabilize the emulsion.

A hydrophilic or hydrophobic ointment: The compositions are formulated with a hydrophobic base (e.g. petrolatum, thickened or gelled water insoluble oils, and the like) and optionally having a minor amount of a water soluble phase. Hydrophilic ointments generally contain one or more surfactants or wetting agents

A microemulsion: These are clear, thermodynamically stable isotropic liquid systems that contain oil, water and surfactants, frequently in combination with a cosurfactant. Microemulsions may be water continuous, oil continuous or bicontinuous mixtures. The formulations may optionally also contain water up to 60% by weight. Higher levels may be suitable in some compositions.

A nanoemulsion: These are isotropic dispersed systems that contain water, oil, and an emulsifier. The system may be an oily system dispersed in an aqueous system, or an aqueous system dispersed in an oily system forming droplets or oily phases of nanometric sizes. Nanoemulsions often have higher loading capacity for lipophilic active ingredients than microemulsions. Hydrophobic and hydrophilic active ingredients can also be formulated in nanoemulsion. Nanoemulsions may be formed by any suitable method known in the art, including high-pressure homogenization, microfluidization, and phase-inversion temperature.

An aerosol foam or spray: The product may be an alcohol/solvent based solution containing an emulsifying wax or an emulsion comprising a discrete phase of a hydrophobic component and a continuous aqueous phase that includes water and optionally one or more polar hydrophilic excipients as well as solvents, co-solvents, surfactants, emulsifiers, and other components. These solvent or emulsion foam concentrates may include water-soluble or water-swellable polymers that help to stabilize the emulsion and corrosion inhibitors to improve compatibility between the formulation and the package. A hydrocarbon, hydrochlorofluorocarbon (HCFC) or chlorofluorocarbon (CFC) aerosol propellant can be added to the solvent or emulsion foam concentrate in packaging designed to maintain pressure until the foam or spray product is dispensed for application.

Solvents

Compositions for use in the present invention may include one or more solvents or co-solvents which modify skin permeation or the activity of other excipients contained in the formulation. Solvents include, but are not limited to acetone, ethanol, benzyl alcohol, butyl alcohol, diethyl sebacate, diethylene glycol monoethyl ether, diisopropyl adipate, dimethyl sulfoxide, ethyl acetate, isopropyl alcohol, isopropyl isostearate, isopropyl myristate, N-methyl pyrrolidione, polyethylene glycol, glycerol, propylene glycol and SD alcohol.

Surfactants

Compositions for use in the present invention may include one or more surfactants or co-surfactants. Surfactants include, but are not limited to short-chain alcohols, alkane diols and triols, alkyl phosphate esters, polyethylene glycols and glycol ethers, pyrrolidine derivatives, bile salts, sorbitan fatty acid esters and polyoxyethylene sorbitan fatty acid esters.

Polymeric Emulsifiers

Compositions for use in the present invention may include one or more polymeric emulsifier. Polymeric emulsifiers can include high molecular weight copolymers of acrylic acid and C₁₀₋₃₀ alkyl acrylate crosslinked with allyl pentaerythritol. Polymeric emulsifiers particularly suitable for use with the methods of the present invention include those marketed under the tradename Pemulen™ and sold by Lubrizol, including Pemulen™ TR-1 NF and Pemulen™ TR-2 NF.

Moisturizers

Compositions for use in the present invention may include one or more moisturizers to increase the level of hydration. The moisturizer can be a hydrophilic material including humectants or it can be a hydrophobic material including emollients. Suitable moisturizers include, but are not limited to: 1,2,6-hexanetriol, 2-ethyl-1,6-hexanediol, butylene glycol, glycerin, polyethylene glycol 200-8000, butyl stearate, cetostearyl alcohol, cetyl alcohol, cetyl esters wax, cetyl palmitate, cocoa butter, coconut oil, cyclomethicone, dimethicone, docosanol, elastomers, ethylhexyl hydroxystearate, fatty acids, glyceryl isostearate, glyceryl laurate, glyceryl monostearate, glyceryl oleate, glyceryl palmitate, glycol distearate, glycol stearate, isopropyl palmitate, isostearic acid, isostearyl alcohol, lanolin, mineral oil, limonene, medium-chain triglycerides, menthol, myristyl alcohol, octyldodecanol, oleic acid, oleyl alcohol, oleyl oleate, olive oil, paraffin, peanut oil, petrolatum, Plastibase-50W, and stearyl alcohol.

Polymers and Thickeners

For certain applications, it may be desirable to formulate a product that is thickened with soluble, swellable, or insoluble organic polymeric thickeners such as natural and synthetic polymers or inorganic thickeners such as acrylates copolymer, carbomer 1382, carbomer copolymer type B, carbomer homopolymer type A, carbomer homopolymer type B, carbomer homopolymer type C, acrylamide/sodium acryloyldimethyl taurate copolymer, carboxy vinyl copolymer, carboxymethylcellulose, carboxypolymethylene, carrageenan, guar gum, xanthan gum, hydroxyethyl cellulose, hydroxypropyl cellulose, microcrystalline wax, and methylcellulose,

Additional Components

Compositions for use in the present invention may be formulated with additional components such as fillers, carriers and excipients conventionally found in cosmetic and pharmaceutical topical products. Additional components including but not limited to antifoaming agents, preservatives (e.g. p-hydroxybenzoic esters, benzyl alcohol, phenylmercury salts, chlorocresol), antioxidants (e.g., BHT, BHA, ascorbic acid, tocopherol, citric acid, propyl gallate, sodium metabisulfite), sequestering agents, stabilizers, buffers, pH adjusting agents (preferably agents which result in an acidic pH, including but not limited to gluconolatone, citric acid, lactic acid, and alpha hydroxyacids), skin penetration enhancers, skin protectants (including but not limited to petrolatum, paraffin wax, dimethicone, glyceryl monoisostearate, isopropyl isostearate, isostearyl isostearate, cetyl alcohol, potassium cetyl phosphate, cetyl behenate and behenic acid), chelating agents, film formers, dyes, pigments, diluents, bulking agents, fragrances, aerosol producing agents and other excipients to improve the stability or aesthetics, may be added to the composition.

Compositions for use in the present invention may be formulated with additional active agents depending on the conditions being treated. Exemplary additional active agents for a combination topical drug product include corticosteroids (e.g., clobetasol, betamethasone, halobetasol, or triamcinolone), beta andrenergic antagonists (e.g., timolol), calcineurin inhibitors (e.g., tacrolimus, or pimecrolimus), methotrexate, or cyclosporine.

In certain embodiments, the topical pharmaceutical composition can include laureth-4, which can function as a skin penetration enhancer.

Administration and Dosage

The present invention includes methods of treating vitiligo to a patient in need thereof, comprising topically applying an effective amount of the composition described herein to the outer epithelial surface of the human body. In certain embodiments, the active ingredient, SHR0302, can be administered in a therapeutically effective amount. In certain embodiments, the amount of SHR0302 can range from about 0.01% w/w to about 5% w/w, or from about 0.1% w/w to about 3% w/w. Exemplary ranges are from about 0.01% w/w to about 2% w/w, or from about 0.01% w/w to about 1.5% w/w, or from about 0.1% w/w to about 1.0% w/w; or 0.1% w/w to about 0.5% w/w. For example, the topical formulation comprises any of the following w/w percents of SHR0302: 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 7%, 1.8%, 1.9%, 1.0%, 2.1%, 2.2%, 2.3%, 2.4%, 2.5%, etc.

In certain embodiments, the pharmaceutical composition is administered topically as a regimen, such as at regular intervals. For example, a topical pharmaceutical composition can be administered once daily, twice daily, thrice daily, once per week, twice per week, three times per week, or four times per week. The pharmaceutical compositions can be administered for a prescribed period of time. For example, a topical pharmaceutical composition can be administered for a period of about two weeks to at least about six months, or until an improvement in skin condition or disease is visually observed. Exemplary periods of time for the treatment regimen include two weeks, one month, six weeks, two months, three months, four months, five months, six months, seven months, eight months, nine months, or one year. In preferred embodiments, the topical pharmaceutical composition is administered twice or thrice daily for a period of at least 3 months, 4 months, 5 months, or 6 months.

The methods of the present invention can further include administering a UVB phototherapy to the subject having vitiligo. In preferred embodiments, the phototherapy is narrowband UVB. The phototherapy can be administered according to The Vitiligo Working Group recommendations for narrowband ultraviolet B light phototherapy treatment of vitiligo. J. Am. Acad. Dermatol. 2017 May; 76(5):879-888.

The phototherapy can be administered to the subject's vitiligo lesions, including lesions of the face, neck, forearms, and/or hands. In various embodiments, the phototherapy is applied at least to the subject's skin section that is afflicted by vitiligo, including vitiligo lesions of the hands, forearms, face, elbows, and/or neck. In various embodiments, the phototherapy is substantially restricted to the subject's skin section that is afflicted by vitiligo. In certain embodiments, the subject's skin that is afflicted by vitiligo is free of all topical products, including for example, a topical pharmaceutical composition, sunscreen, moisturizers, makeup for 30-minutes prior to beginning a phototherapy session (except mineral oil if needed), because of the possibility of deactivation or interference with transmission of narrowband UVB.

In certain embodiments, the phototherapy is delivered by a full body-sized unit or a smaller unit or a handheld device. In yet other embodiments, the phototherapy is administered using a home phototherapy unit, such as the Daavlin Series 1 NBUVB Home Phototherapy Unit. In certain embodiments, the patient is administered a dose about 10 to 1,000 mJ/cm² of nbUVB is administered to the patient. In certain embodiments, the phototherapy is administered in a dose of 10 to 5000 mJ/cm² of nbUVB. In certain embodiments, the phototherapy is administered in a dose of 25 to 1,000 mJ/cm² of nbUVB. In certain embodiments, the phototherapy is administered in a dose of 25 to 500 mJ/cm² of nbUVB. In certain embodiments, the phototherapy is administered in a dose of 25 to 250 mJ/cm² of nbUVB. In certain embodiments, the phototherapy is administered in a dose of 200 mJ/cm². In certain embodiments, the subject is administered a tapered dose of phototherapy over time once they achieve complete repigmentation of the treated area. For example, during the first month the phototherapy is administered twice weekly, during the second month the phototherapy is administered once weekly, and during the third and fourth months the phototherapy is administered every other week.

In certain embodiments, the phototherapy may be applied for between 1 second and 16 minutes and 30 seconds for each area treated. For example, the phototherapy may be applied for 1 second, 30 seconds, 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 16 minutes and 10 seconds, or 16 minutes and 30 seconds. The treatment dose may be determined based on patient feedback (e.g., skin sensitivity) as well as characteristics of the dressing (e.g., how much UV will penetrate to skin, size of the treatment area, etc.) and characteristics of the UV source (e.g., how much energy the device will or is capable of delivering).

In certain embodiments, the phototherapy is administered as a regimen, such as at regular intervals. For example, the phototherapy can be administered once daily, once per week, twice per week, three times per week, four times per week, once bi-weekly, or once monthly. In certain embodiments, the phototherapy is administered three times a week with at least 1 day between treatments. For example, the phototherapy is administered Monday-Wednesday-Friday, or Tuesday-Thursday-Saturday. The phototherapy can be administered for a prescribed period of time, including, for a period of about two weeks to at least about six months, or until an improvement in skin condition or disease is visually observed. Exemplary periods of time for the treatment regimen include two weeks, one month, six weeks, two months, three months, four months, five months, six months, seven months, eight months, nine months, or one year. In preferred embodiments, the phototherapy is administered twice or thrice weekly for a period of between one and four months.

The methods of the present invention can further include treatment of facial vitiligo, including patients having facial vitiligo lesions. The methods described herein can alleviate the symptoms of vitiligo, including repigmenting a subject's vitiligo lesions, including facial lesions. The methods described herein can further reduce the subject's facial vitiligo area and severity index (F-VASI), face and neck vitiligo area and severity index (F/N-VASI), forearm vitiligo area and severity index (forearm-VASI).

The following examples illustrate certain embodiments of the invention without limitation.

### EXAMPLES

While various embodiments have been described above, it should be understood that they have been presented by way of example only, and not limitation. Thus, the breadth and scope of the present disclosure should not be limited by any of the above-described exemplary embodiments. Moreover, any combination of the above-described elements in all possible variations thereof is encompassed by the disclosure unless otherwise indicated herein or otherwise clearly contradicted by context.

Experimental Example 1

A 35-kg batch of ARQ-252 cream 0.3% (SHR0302 free base equivalent) was manufactured and filled into sealed aluminum tubes (¾″×3¾″ #16 cap). The first step in preparing the cream was to make three blends or “Phases” of the Cream components for use later during manufacturing of the cream. The three preparations are the “25% Trolamine Stock Solution,” the “Internal Phase” and the “API Phase” of the ARQ-252 Cream. In a 1-liter glass beaker 375.97 grams of water was combined with 125.00 grams of Trolamine, NF and mixed. This “25% Trolamine Stock Solution” was labeled and set aside for use to adjust pH of the cream later. In a 7-liter stainless steel stock pot the “Internal Phase” of the cream was prepared, labelled, and set aside for later addition to the main reaction vessel during product manufacture. The “Internal Phase” was made by combining and mixing 2471.87 grams Cyclomethicone, NF, 700.61 grams of Dow Corning ST-Elastomer 10, 35.22 grams D-limonene and 349.77 grams of Dimethicone, 350 cst. The “API Phase” was made by blending 10.56 kgs Dimethyl Sulfoxide, USP, 701.77 grams of Benzyl Alcohol, NF and 17.60 grams of Butylated Hydroxytoluene, NF in a 20-liter Stainless Steel Stock Pot and then dissolving 132.25 grams of SHR0302 sulfate salt.

In the 36-liter stainless steel stock pot 5257.89 grams of Propylene Glycol (PG), 5253.18 grams of Polyethylene Glycol 200 (PEG 200), 279.57 grams of Carbomer Copolymer (Pemulen™ TR-1) and 528.17 grams of Carbomer Homopolymer (Carbopol 974P) were combined and mixed. In a 20-liter stainless steel stock pot side vessel, 8884.65 grams of water was combined with 17.48 grams of Edetate Disodium, Diydrate USP was mixed to uniformity. The entire contents of this 20-liter stainless steel stock pot was added to the 36-liter stainless steel stock pot of PG, PEG 200 and the two Carbomers with constant mixing. The PG, PEG 200, Carbomer Copolymer, Carbomer Homopolymer, water and Edetate Disodium-Dihydrate is then transferred into the 50-liter jacketed main reaction vessel that is equipped with anchor mixing.

The “Internal Phase” is added to the main reaction vessel with continuous mixing for not less than 5 minutes. After homogeneous, the “API Phase” is slowly added to the main reaction vessel with continuous mixing until the API phase is well incorporated and for not less than 5 minutes. After the API phase addition and mixing is complete, insert a homogenizer and homogenize to form a uniform white cream, for at least 60 minutes. Take a pH sample, calculate the appropriate amount of “25% Trolamine Stock Solution” to add to adjust to the target pH value of 5.5. If necessary, repeat this process of testing the pH of the cream and adding 25% trolamine until the pH value of the cream is 5.5±0.4 pH units.

Table 1 provides an exemplary topical pharmaceutical composition and vehicle for use with the methods disclosed herein.

TABLE 1

| | ARQ-252 | ARQ-252 |
|---|---|---|
| Ingredient | 0.3% Cream | Vehicle |
| SHR0302 | 0.30% w/w | — |
| Dimethyl Sulfoxide (DMSO) | 30.0% w/w | 30.0% w/w |
| Propylene Glycol | 15% w/w | 15% w/w |
| Polyethylene Glycol 200 | 15% w/w | 15% w/w |
| Benzyl Alcohol | 2.0% w/w | 2.0% w/w |
| Cyclomethicone | 7.0% w/w | 7.0% w/w |
| ST-Elastomer 10 | 2.0% w/w | 2.0% w/w |
| Dimethicone | 1.0% w/w | 1.0% w/w |
| Pemulen TR 1 | 0.8% w/w | 0.8% w/w |
| Carbopol 974P | 1.5% w/w | 1.5% w/w |
| EDTA | 0.05% w/w | 0.05% w/w |
| BHT | 0.05% w/w | 0.05% w/w |
| D-Limonene | 0.1% w/w | 0.1% w/w |
| Trolamine (25% solution | q.s. ad | q.s. ad |
| to adjust pH) | pH 5.5 | pH 5.5 |
| Purified Water | q.s. ad 100% | q.s. ad 100% |
| Total | 100% | 100% |

### Example 2

Example 2 provides two additional exemplary topical pharmaceutical compositions for use in the methods described herein.

TABLE 2

| | Formulation | Formulation |
|---|---|---|
| | # 1 | # 2 |
| | (2019-048-55) | (2019-048-57) |
| Ingredients | % w/w | % w/w |
| SHR0302 | 0.5 | 0.5 |
| DMSO | 35.0 | 35.0 |
| Laureth-4 | — | 4.0 |
| Butylated | 0.05 | 0.05 |
| Hydroxytoluene | | |
| Benzyl Alcohol | 2.0 | 2.0 |
| Propylene Glycol | 15.0 | 12.5 |
| PEG 200 | 15.0 | 12.5 |
| Cyclomethicone | 7.0 | 7.0 |
| Dimethicone (350 | 1.0 | 1.0 |
| cst) | | |
| ST-Elastomer 10 | 2.0 | 2.0 |
| Pemulen TR 1 | 0.8 | 0.8 |
| Carbopol 974P | 1.5 | 1.5 |
| Purified Water | Q.S to 100 | Q.S to 100 |
| 25% Trolamine | pH to 5.5-5.9 | pH to 5.5-5.9 |
| 10% (w/v) HCl | pH to 5.5-5.9 | pH to 5.5-5.9 |

### Example 3

Example 3 provides three additional exemplary topical pharmaceutical compositions for use in the methods described herein.

TABLE 3

| | Formulation | Formulation | Formulation |
|---|---|---|---|
| | # 3 | # 4 | # 5 |
| | (BR18034A) | (2019-006-27) | (2019-048-59) |
| Ingredients | % w/w | % w/w | % w/w |
| SHR0302 | 0.3 | 0.3 | 0.3 |
| N-Methyl-2 | 20.0 | 20.0 | 20.0 |
| Pyrriolidone | | | |
| Laureth-4 | 4.0 | 0.1 | 4.0 |
| Butylated | 0.05 | 0.05 | 0.05 |
| Hydroxytoluene | | | |
| Methylparaben | 0.2 | 0.2 | 0.2 |
| Propylparaben | 0.05 | 0.05 | 0.05 |
| Crodafos CES | 10.0 | 10.0 | 10.0 |
| Isopropyl | 5.0 | 5.0 | 5.0 |
| Palmitate | | | |
| White Petrolatum | 5.0 | 5.0 | 5.0 |
| (Protopet 1 S) | | | |
| Propylene Glycol | 15.0 | 15.0 | 15.0 |
| PEG 200 | 15.0 | 15.0 | 15.0 |
| 1N Sodium | 3.5 | 3.5 | 3.5 |
| Hydroxide | | | |
| Xanthan Gum | — | 0.2 | 0.2 |
| Purified Water | Q.S to 100 | Q.S to 100 | Q.S to 100 |
| 10% Sodium | pH to 5.5-5.9 | pH to 5.5-5.9 | pH to 5.5-5.9 |
| Hydroxide | | | |

### Example 4

A Proof of Concept, parallel group, double blind, vehicle-controlled study of the safety and efficacy of a 0.3% SHR0302 topical cream of Example 1 (ARQ-252 cream 0.3%) was designed for subjects with non-segmental facial vitiligo. Subjects with non-segmental facial vitiligo applied either ARQ-252 0.3% cream BID or vehicle cream BID to all affected areas of vitiligo on the face, neck, hands, forearms, and elbows. Product was applied to all affected, depigmented areas and, where anatomically possible, treatment extended approximately 3 cm from the border all around. These same subjects also used the Daavlin Series 1 NBUVB Home Phototherapy Unit to the entire face and areas affected on the neck, hands, forearms, and elbows up to 3×/week per a standardized phototherapy protocol. One patient applied the ARQ-252 cream 0.3% twice daily in combination with phototherapy using the Daavlin Series 1 NBUVB Home Phototherapy Unit. One patient applied twice-a-day vehicle cream (same composition as ARQ-252 cream except the SHR0302 active has been replaced with water) in combination phototherapy using the Daavlin Series 1 NBUVB Home Phototherapy Unit.

The two patients were assessed by measures including Facial Vitiligo Area and Severity Index (F-VASI), Facial Body Surface Area (F-BSA), Facial/Neck BSA (F/N-BSA), BSA (Hands, Forearms, Elbows), Facial/Neck VASI (F/N-VASI), VASI of the forearms and elbow (FOREARM-VASI) and VASI of the hands only (HAND-VASI). The area “Face” is defined as including the area on the forehead to the original hairline, on the cheek to the jawline vertically to the jawline and laterally from the corner of the mouth to the tragus. The area “Face” does not include surface area of the lips, scalp, ears, or neck but will include the eyelids and nose. VASI is a quantitative clinical tool that is based on a composite estimate of the overall area of vitiligo patches at baseline and the degree of macular repigmentation within these patches over time. The reliability and validity of F-VASI instruments as measures of treatment efficacy have been confirmed. (Rosmarin, 2020). BSA affected with vitiligo will be assessed as a percentage of the total BSA, and will be determined to the nearest 0.01% using, as guides, the palm plus 5 digits, with fingers tucked together and thumb tucked to the side (handprint), as 1% BSA and the thumb as 0.1% BSA. The degree of depigmentation for each vitiligo involvement site was determined and estimated to the nearest of the following percentages: 0, 10%, 25%, 50%, 75%, 90%, or 100%. At 100% depigmentation, no pigment is present; at 90%, specks of pigment are present; at 75%, the depigmented area exceeds the pigmented area; at 50%, the depigmented and pigmented area are equal; at 25%, the pigmented area exceeds the depigmented area; at 10%, only specks of depigmentation are present. The VASI was then derived by multiplying the values assessed for the vitiligo involvement by the percentage of affected skin for each site on the face and summing the values of all sites together. The percentage of vitiligo involvement was estimated in hand units by the same investigator during the entire course of the study. As seen in Table 4, when the JAK inhibitor SHR0302 is applied topically in combination with narrowband UVB phototherapy vitiligo lesions are dramatically repigmented compared to topical treatment of vehicle cream (no SHR0302) combined with narrowband UVB phototherapy. This improved therapy regimen was most efficacious in treating vitiligo lesions of the face and neck.

TABLE 4

| | Twice daily ARQ-252 cream 0.3% | | Twice daily ARQ-252 vehicle | |
|---|---|---|---|---|
| | (example 1) and Daavlin Series 1 | | (Table 1) and Daavlin Series 1 | |
| | NBUVB Phototherapy (3X/week) | | NBUVB Phototherapy (3X/week) | |
| | Baseline | Week 12 | Baseline | Week 12 |
| F-VASI | 0.27 | 0.1 | 0.9 | 0.6 |
| F/N-VASI | 0.27 | 0.1 | 1.5 | 0.675 |
| FOREARM- | 0.27 | 0.18 | 0.225 | 0.375 |
| VASI | | | | |
| HAND-VASI | 0.45 | 0.36 | 0.45 | 0.3 |
| Evaluation Date | 24 Mar. 2021 | 10 Jun. 2021 | 16 Mar. 2021 | 18 Jun. 2021 |

| | | |
|---|---|---|
| Change F-VASI | 0.17 | 0.3 |
| % Change in | 63% improvement | 33% improvement |
| VASI | | |

The foregoing description has been presented for purposes of illustration and description. This description is not intended to limit the invention to the precise form disclosed. Persons of ordinary skill in the art will appreciate that modifications and substitutions of the basic inventive description may be made.

What is claimed is:

1. A method of treating vitiligo in a subject in need thereof comprising:
(a) topically administering to the subject a therapeutically effective amount of SHR0302 or a pharmaceutically acceptable salt thereof; and
(b) administering to the subject an ultraviolet B phototherapy.

2. The method of claim 1, wherein the SHR0302 or salt thereof is administered in a pharmaceutical composition comprising about 0.10% w/w to about 0.50% w/w of SHR0302.

3. The method of claim 2, wherein the pharmaceutical composition further comprises a solvent.

4. The method of claim 2, wherein the pharmaceutical formulation further comprises an antioxidant, a preservative, an emulsifier, a moisturizer, or a thickener.

5. The method of claim 2, wherein the pharmaceutical composition is selected from the group consisting of an oil-in-water emulsion, a water-in-oil emulsion, a microemulsion, a nanoemulsion, a foam, a spray, a hydrophilic ointment, or a hydrophobic ointment.

6. The method of claim 2, wherein the pharmaceutical composition is administered at least once daily for at least one week.

7. The method of claim 2, wherein the pharmaceutical composition is administered twice daily for at least one month.

8. The method of claim 1, wherein the ultraviolet B phototherapy is administered to the subject between one and three times per week.

9. The method of claim 1, wherein the ultraviolet B phototherapy is narrowband ultraviolet B phototherapy.

10. A method of treating vitiligo lesions in a subject in need thereof comprising:
(a) topically administering to the subject a therapeutically effective amount of SHR0302 or a pharmaceutically acceptable salt thereof; and
(b) administering to the subject an ultraviolet B phototherapy.

11. The method of claim 10, wherein the method of treatment repigments the subject's vitiligo lesions.

12. The method of claim 10, wherein the method of treatment reduces at least one of the subject's facial vitiligo area and severity index (F-VASI), face and neck vitiligo area and severity index (F/N-VASI), and forearm vitiligo area and severity index (forearm-VASI).

13. The method of claim 10, wherein the SHR0302 or salt thereof is administered in a pharmaceutical composition comprising about 0.10% w/w to about 0.50% w/w of SHR0302.

14. The method of claim 13, wherein the pharmaceutical composition further comprises a solvent.

15. The method of claim 13, wherein the pharmaceutical formulation further comprises an antioxidant, a preservative, an emulsifier, a moisturizer, or a thickener.

16. The method of claim 13, wherein the pharmaceutical composition is selected from the group consisting of an oil-in-water emulsion, a water-in-oil emulsion, a microemulsion, a nanoemulsion, a foam, a spray, a hydrophilic ointment, or a hydrophobic ointment.

17. The method of claim 13, wherein the pharmaceutical composition is administered at least once daily for at least one week.

18. The method of claim 13, wherein the pharmaceutical composition is administered twice daily for at least one week.

19. The method of claim 10, wherein the ultraviolet B phototherapy is administered to the subject between one and three times per week.

20. The method of claim 10, wherein the ultraviolet B phototherapy is narrowband ultraviolet B phototherapy.

21. A method of repigmenting vitiligo lesions of a subject in need thereof comprising:
(a) topically administering to the subject a therapeutically effective amount of SHR0302 or a pharmaceutically acceptable salt thereof; and
(b) administering to the subject an ultraviolet B phototherapy,
wherein the method of treatment repigments the subject's vitiligo lesions.

22. The method of claim 21, wherein the method of treatment reduces at least one of the subject's facial vitiligo area and severity index (F-VASI), face and neck vitiligo area and severity index (F/N-VASI), and forearm vitiligo area and severity index (forearm-VASI).

23. The method of claim 21, wherein the SHR0302 or salt thereof is administered in a pharmaceutical composition comprising about 0.10% w/w to about 0.50% w/w of SHR0302.

24. The method of claim 23, wherein the pharmaceutical composition further comprises a solvent.

25. The method of claim 23, wherein the pharmaceutical formulation further comprises an antioxidant, a preservative, an emulsifier, a moisturizer, or a thickener.

26. The method of claim 23, wherein the pharmaceutical composition is selected from the group consisting of an oil-in-water emulsion, a water-in-oil emulsion, a microemulsion, a nanoemulsion, a foam, a spray, a hydrophilic ointment, or a hydrophobic ointment.

27. The method of claim 23, wherein the pharmaceutical composition is administered at least once daily for at least one week.

28. The method of claim 23, wherein the pharmaceutical composition is administered twice daily for at least one month.

29. The method of claim 21, wherein the ultraviolet B phototherapy is administered to the subject between one and three times per week.

30. The method of claim 21, wherein the ultraviolet B phototherapy is narrowband ultraviolet B phototherapy.
